(19) United States
(12) Patent Application Publication
(10) Pub. No.: US 2024/0293150 A1
(43) Pub. Date: Sep. 5, 2024

(54) STEERABLE ROBOTIC NEEDLES WITH TUNABLE STIFFNESS SEGMENTS FOR LARGE CURVATURE MANEUVERS

(71) Applicant: Wanliang Shan, Jamesville, NY (US)

(72) Inventor: Wanliang Shan, Jamesville, NY (US)

(73) Assignee: Syracuse University, Syracuse, NY (US)

(21) Appl. No.: 18/573,512

(22) PCT Filed: Jun. 24, 2022
(86) PCT No.: PCT/US22/34814

§ 371 Date: Dec. 22, 2023

Related U.S. Application Data
(60) Provisional application No. 63/215,147, filed on Jun. 25, 2021.

(51) Int. Cl. A61B 17/34; A61B 10/04; A61B 17/00; A61B 34/30; A61L 31/12; A61L 31/14

(52) U.S. Cl. CPC A61B 17/3421; A61B 10/04; A61B 34/30; A61L 31/128; A61L 31/14; A61B 2010/045; A61B 2017/00318; A61B 2017/00867; A61B 2017/00964; A61B 2034/301

(57) ABSTRACT

A steerable robotic needle formed from a compliant wire that is surrounded by an inner layer having one or more tunable regions positioned in predetermined locations and an inert region extending over the rest of the wire. The tunable region changes stiffness in response to an external trigger, such as heating via electrical energy, so that the needle can bend more readily in the predetermined location. The inner layer may be encapsulated in an elastomeric tube to prevent heat from reaching the surrounding environment. The resulting change in stiffness allows the needle to perform maneuvers in a body that require a larger curvature.

### CROSS-REFERENCE TO RELATED APPLICATIONS

The present application claims priority to U.S. Provisional App. No. 63/215,147, filed on Jun. 25, 2021.

### BACKGROUND OF THE INVENTION

### 1. Field of the Invention

The present invention relates to surgical needles and, more specifically, to a needle having one or more predetermined sections with tunable stiffness to allow the needle to change bending shape and navigation trajectory during use.

### 2. Description of the Related Art

Early detection through biopsy is key for cancer prevention and treatment, while minimally invasive surgeries with localized drug delivery and/or ablation can significantly improve patient experience and treatment efficacy. Existing designs of compliant robotic needles are not, however, capable of achieving maneuvers that require a large curvature and thus limit the extent to which such needles can be used. As a result, there is a need in the art for robotic needles that can change shape during a procedure to allow the need to perform maneuvers that require a larger curvature.

### BRIEF SUMMARY OF THE INVENTION

A compliant robotic needle formed from a compliant wire that is surrounded by a multi-functional inner layer having at least one region with tunable stiffness. In response to an external trigger, such as heating, the region with the tunable stiffness will allow the needle to bend into a larger curvature. The tunable stiffness region is formed from a smart material that can change in stiffness in response to heating from a first stiffness comparable to the rest of the inner layer to a second stiffness that allows the needle to bend into a larger curvature. Thus, the stiffness of the region can be adjusted during use so that the needle can perform maneuvers that require a larger curvature, such as biopsies, localized drug delivery, and ablation for treatment of cancer and many other diseases.

In an embodiment, the invention is a steerable needle formed by a wire extending a predetermined length, a first region of a first material surrounding a first portion of the wire, and a second region of a second material that is different than the first material surrounding a second portion of the wire, and an elastomer surrounding the first region and the second region, The second material is characterized by a rigidity that is variable in response to an external stimulus. For example, the external stimulus may be heat induced by applying an electrical current to the wire. The second material may be a composite consisting of an elastomeric matrix having a plurality of particles that are rigid at a first temperature, and flexible at a second, higher temperature.

The plurality of particles may be formed from a low melting point alloy (LMPA) with melting temperature roughly between 25° C. and 100° C. The LMPA may be selected from the group including Field's Metal that melts at 62° C., and bismuth metal alloys such as Cerrolow 117 that melts at 47.2° C. The plurality of particles may be formed by a mixture of nickel coated carbon fibers and LMPA particles. The plurality of particles may be formed by a mixture of silver coated carbon fibers and LMPA particles. The second material may be an elastomeric matrix and a tunable foam matrix. The elastomeric matrix may be selected from the group consisting of polydimethylsiloxanes (PDMS), platinum-catalyzed silicones, polyurethanes, silicone polymers, and combinations thereof. The tunable foam matrix may be made of a LMPA. The external stimulus may be an activation voltage coupled to the wire that is sufficient to induce an increase in temperature in the wire. The wire may be nitinol. The wire may be a polymer.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a high level schematic of a robotic needle according to the present invention for use in a surgical procedure.

FIG. 2 is a detailed schematic of the composition of a needle that can change shape during use according to the present invention.

FIG. 3 is a schematic of the composition of an exemplary needle according to the present invention.

### DETAILED DESCRIPTION OF THE INVENTION

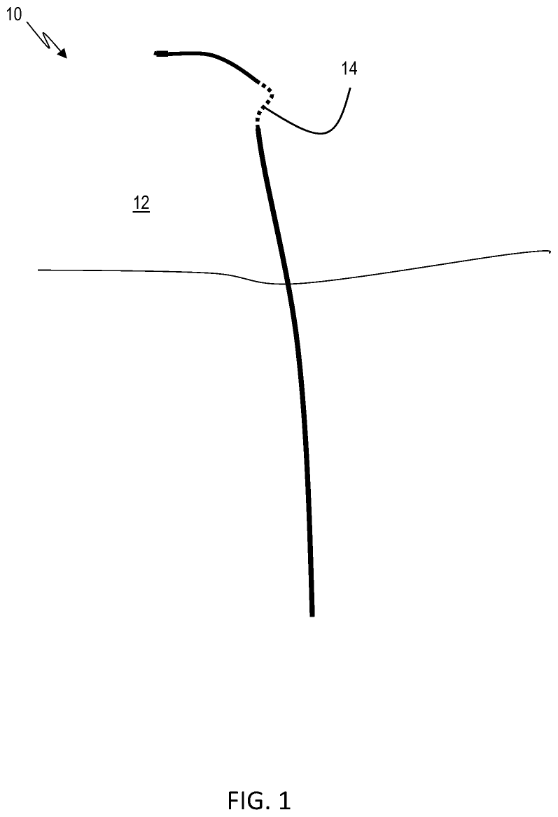

Referring to the figures, wherein like numeral refer to like parts throughout, there is seen in FIG. 1 a compliant robotic needle 10 that can change shape during use for performing maneuvers in a body 12 of a patient that require a larger curvature through the use of a tunable segment 14 having a variable bending rigidity. Needle 10 may thus for biopsies and localized drug delivery in all locations, thereby significantly reducing cost for patients and thus making it accessible to low-income populations.

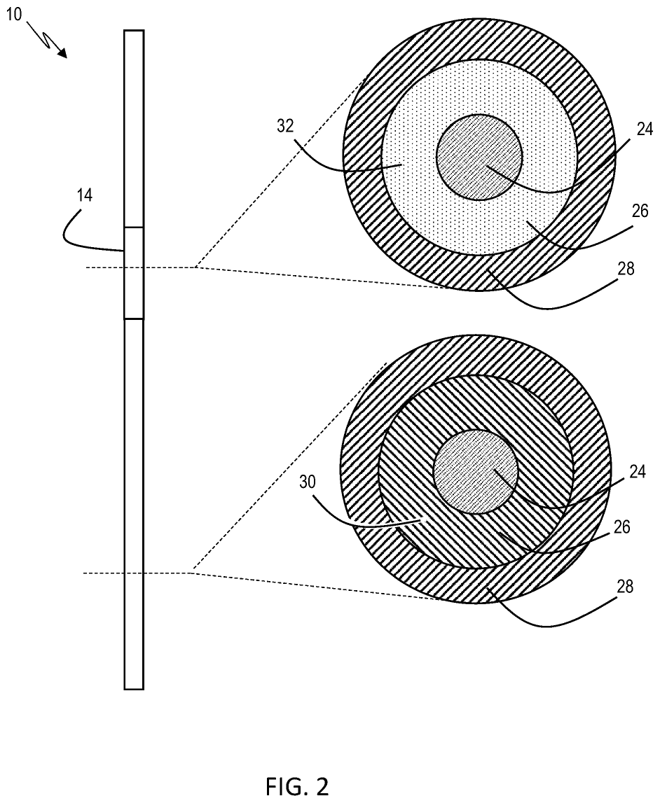

As seen in FIG. 2, needle 10 may be formed from a thin rod or wire 24 that is surrounded by a first, variable layer 26, and an outer elastomeric tube 28. Variable layer 26 comprises at least one inert region 30 formed from a plastic material that is inert to heating and extends along the length of wire 24. Variable layer 26 further comprises at least one tunable region 32 that comprises of a smart material having a rigidity that may be tuned during use. Thus, the positioning of inert region 30 and tunable region 32 can be used to control where wire 24 will be allowed to curve or bend more easily when tunable region 32 is triggered so that the rigidity or stiffness of the smart material changes.

As demonstrated in FIG. 1, dynamic control of the buckling shape of needle 10 and thus its trajectory in a substance can be achieved through applying load f from one end of the needle, where maneuvers of larger curvatures can be achieved by dynamically tuning the bending rigidity, κ, of tunable region 32 of needle 10. As κ directly dictates the critical buckling load f_{c} as f_{c}∝κ^{1/2}, it follows that, if a segment suddenly softens, a straight thin rod will buckle under much smaller loading, and that a buckled thin rod will buckle further.

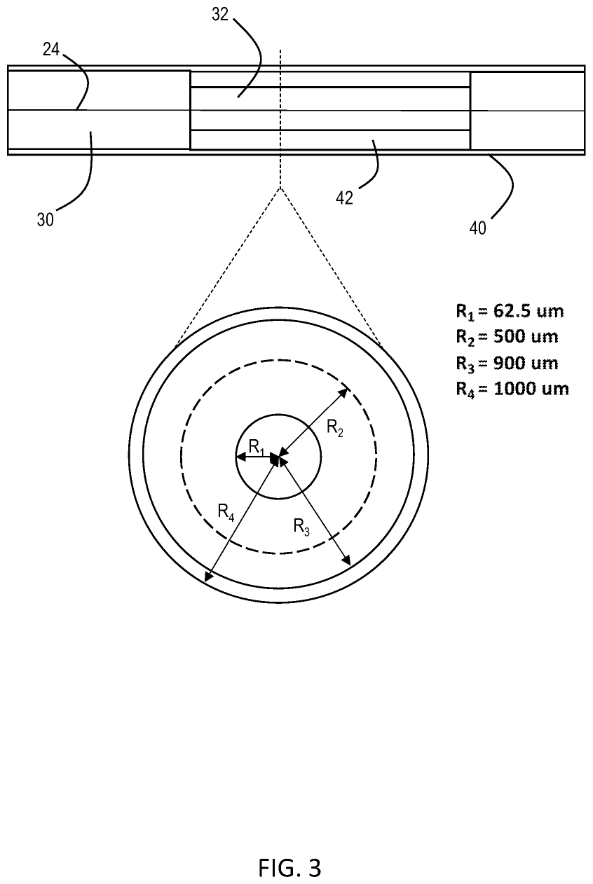

As further seen in FIG. 3, needle 10 has an axisymmetric multi-layered design. As an example, needle 10 has an inner core formed from a superelastic Nitinol wire 24 throughout its length, a middle layer 32 formed from an elastomeric smart material with tunable stiffness, or a plastic inert to heating but with similar rigidity as the smart materials when not activated, and an outmost layer 40 formed from soft elastomers or plastics that insulate the surrounding area when needle 10 is heated to activate the change in stiffness of the smart material. This arrangement insulates heat during activation from the surrounding media. This design also guarantees that the overall thin composite rod can go back to its original straight status when unloaded and heated again. The effective bending rigidity, \tilde{κ} of the composite thin rod seen in FIG. 2 can be calculated as follows:

$${k}^{\text{?}} = {E}_{1} {I}_{1} + {E}_{2} {I}_{2} + {E}_{3} {I}_{3} = \frac{π}{2} ( {E}_{1} {r}_{1}^{4} + {E}_{2} {r}_{2}^{4} - {E}_{2} {r}_{1}^{4} + {E}_{3} {r}_{3}^{4} - {E}_{3} {r}_{2}^{4} ) = {E}_{1} {I}_{1} ( 1 + \frac{{E}_{2} {r}_{2}^{4}}{{E}_{1} {r}_{1}^{4}} - \frac{{E}_{2}}{{E}_{1}} + \frac{{E}_{3} {r}_{3}^{4}}{{E}_{1} {r}_{1}^{4}} - \frac{{E}_{3} {r}_{2}^{4}}{{E}_{1} {r}_{1}^{4}} ) = {kE}_{1} {I}_{1} .$$

$$\text{?} \text{indicates text missing or illegible when filed}$$

Here, k is a coefficient to describe how much \tilde{κ} is in terms of the bending rigidity of the Nitinol wire E₁I₁. For a 125 μm thick Nitinol wire available from Amazon Inc., E₁=60 GPa, r₁=62.5 μm. For the novel smart composite with tunable stiffness, E₂=100 MPa, and r₂ can be 700 μm. After activation E₂=1 MPa. Lastly, for a typical elastomer tube, E₃=1 MPa, and r₃ can be 900 μm. Using this simplified example, \tilde{κ}=27.7 before activation, and \tilde{κ}=1.72 after activation, which is 16× change. Since the radius is to the power of 4 in Eqn. 1, slight change in the geometry can lead to even higher bending rigidity change. Note that under the current calculation the total diameter of the composite needle is 1.6 mm, which is comparable with the size of currently used robotic needles. For practical biomedical use, these dimensions can be scaled down further to design sub-millimeter needles. Undesirable hyperthermia during activation can be well addressed by judicial mechanical design assisted with heat transfer simulations. For ablation purpose, the insulating outside layer made of elastomer or plastics at the ablation segment can be replaced with elastomer-based smart composites with high thermal conductivity to facilitate heat diffusion.

Acceptable smart materials for tunable region 32 may comprise an elastomeric matrix with tunable particles. The elastomeric matrix should have a low elastic modulus (e.g., 10 kPa-1 MPa), such as PDMS, Ecoflex® (a platinum-catalyzed silicone material), polyurethane, Elastosil® (silicone rubber-based materials consisting essentially of silicone polymers and fillers), and combinations thereof. Tunable particles are particles that can be capable of rigidity tuning, that is, they are capable of converting from being rigid to being flexible or vice versa. The tunable particles can be orders of magnitude more rigid (stiffer) than the elastomeric matrix at room temperature. Materials capable of rigidity tuning include materials that are susceptible to heat such that the material softens (e.g., the Young's modulus of the material is reduced) when exposed to a particular temperature or electrical current. In some embodiments, the tunable particles comprise a material that is rigid at room temperature, but that becomes soft/flexible when heated to a temperature above either the material's glass transition temperature and/or its melting point. In some embodiments, the material can be less rigid at room temperature and more rigid at temperatures below room temperature. Exemplary tunable particle materials include, but are not limited to, LMPA, such as Field's Metal (having a melting point of 62° C.), as well as Bismuth-based alloy metals (e.g., Cerrolow 117 having a melting point of 47.2° C.). Due to their extremely low electrical resistivity (˜2×10⁻⁶ Ω·m), LMPAs are suitable for micro-scale embodiments of the composites, where fast activation by a small sized power supply, such as a battery, is possible.

Acceptable smart materials for tunable region 32 may also comprise a composite with a plurality of conductive fibers and conductive particles with tunable stiffness. For example, the composite can comprise a plurality of nickel coated carbon fibers (NCCF) of approximately 0.1 mm length, in addition to LMPA particles. In other embodiments, silver coated carbon fibers (SCCF) can be used for the fibers. In some embodiments, the fiber length can be between approximately a few hundred nanometers to 500 μm. LMPA fibers can also be used in conjunction with LMPA particles to allow for higher stiffness change and electrical conductivity.

Acceptable smart materials for tunable region 32 may further comprise a composite having a bicontinuous network of two matrices, an elastomeric matrix and a tunable foam matrix. Elastomeric matrix can comprise one or more elastomers. The elastomers can have a low elastic modulus (e.g., 10 kPa-1 MPa). Exemplary elastomers include, but are not limited to, PDMS, Ecoflex® (a platinum-catalyzed silicone material), Polyurethane, Elastosil® (silicone rubber-based materials consisting essentially of silicone polymers and fillers), and combinations thereof. The tunable foam matrix can be capable of rigidity tuning, that is, it is capable of converting from being rigid to being flexible or vice versa. The tunable foam matrix can be orders of magnitude more rigid (stiffer) than the elastomeric matrix at room temperature, such as LMPA. In particular disclosed embodiments, materials capable of rigidity tuning include materials that are susceptible to heat such that the material softens (e.g., the Young's modulus of the material is reduced) when exposed to a particular temperature or electrical current. In some embodiments, the tunable particles comprise a material that is rigid at room temperature, but that becomes soft/flexible when heated to a temperature above the material's melting point. In some embodiments, the material can be less rigid at room temperature and more rigid at temperatures below room temperature.

The tunable foam matrix can comprise a LMPA. The stiffness of the composite can be tuned by inducing phase changes in the LMPA component. Below the melting point of the LMPA, the composite behaves like a solid metal and is stiff. Above the melting point, the LMPA will be liquid, therefore the mechanical properties of the polymer foam dominate the composite's mechanical properties, and the composite behaves like a soft material. Exemplary LMPAs include Cerrolow 117 (having a melting point of 47.2° C.), and Field's Metal (having a melting point of 62° C.).

Needle 10 is coupled to an electrical current to heat smart materials for tunable region 32. The electrical current can be provided at a particular activation voltage, which can be selected based on the structural features of the composite as described herein. In some embodiments, the activation voltage can be applied repeatedly and intermittently using a battery. The composite can be exposed to the electrical current for a sufficient amount of time as to heat the entire composite. In particular, the amount of time needed to heat the composite can be increased or reduced by varying the activation voltage used. Higher activation voltages utilize less heating time, whereas lower activation voltages utilize more heating time.

An exemplary heat transfer simulation has been conducted to investigate the practicality of the design in FIG. 3. Note that except for thermal ablation purpose, temperature at the composite wire surface needs to be controlled carefully, as human cells die at 42° C. Multiphysics Ansys simulation results show that the activation can be finished within 1 second using a pulse of high power, but the heat needs to be absorbed by another melting material (e.g. wax with 40° C. melting point such that the surface temperature of the composite wire is under 42° C. The preliminary simulation also shows that the Cerrolow 117 component in the smart material constitutes ˜80% of the activation barrier, which justifies the adoption of smart materials with less LMPA volume fraction and better properties. With inclusion of wax 42 in tunable region 32, as seen in FIG. 3, the stiffness change of the smart segment will change. For design of real needles, the nitinol wire can be replaced with a much softer shape memory polymer wire (E˜1 GPa), or removed altogether if the specific application requires it.

What is claimed is:

1. A steerable needle, comprising:
a wire extending a predetermined length;
a first region of a first material surrounding a first portion of the wire;
a second region of a second material that is different than the first material surrounding a second portion of the wire, wherein the second material is characterized by a rigidity that is variable in response to an external stimulus; and
an elastomer surrounding the first region and the second region.

2. The steerable needle of claim 1, wherein the external stimulus is heat.

3. The steerable needle of claim 1, wherein the second material comprises an elastomeric matrix having a plurality of particles that are rigid at a first temperature, and flexible at a second, higher temperature.

4. The steerable needle of claim 3, wherein the plurality of particles are formed from a low melting point alloy.

5. The steerable needle of claim 4, wherein the low melting point alloy is selected from the group consisting of Field's Metal and Cerrolow 117.

6. The steerable needle of claim 1, wherein the plurality of particles are formed by a mixture of nickel coated carbon fibers and low melting point alloy particles.

7. The steerable needle of claim 1, wherein the plurality of particles are formed by a mixture of silver coated carbon fibers and low melting point alloy particles.

8. The steerable needle of claim 1, wherein the second material comprises an elastomeric matrix and a tunable foam matrix.

9. The steerable needle of claim 8, wherein the elastomeric matrix is selected from the group consisting of polydimethylsiloxanes, platinum-catalyzed silicones, polyurethanes, silicone polymers, and combinations thereof.

10. The steerable needle of claim 8, wherein the tunable foam matrix includes a low melting point alloy.

11. The steerable needle of claim 8, wherein the low melting point alloy is selected from the group including Field's Metal and Cerrolow 117.

12. The steerable needle of claim 1, wherein the external stimulus comprises an activation voltage coupled to the wire that is sufficient to induce an increase in temperature in the wire.

13. The steerable needle of claim 1, wherein the wire comprises nitinol.

14. The steerable needle of claim 1, wherein the wire comprises a polymer.
